(19) United States
(12) Patent Application Publication
(10) Pub. No.: US 2010/0021570 A1
(43) Pub. Date: Jan. 28, 2010

(54) Smoking Cessation Treatment By Reducing Nicotine Cravings, Apetite Suppression, And Altering The Perceived Taste Of Tobacco Smoke

(76) Inventor: Harlan Clayton Bieley, West Palm Beach, FL (US)

Correspondence Address: JOHN C. SMITH, ESQ., 2499 GLADES ROAD, SUITE 113, BOCA RATON, FL, 33431, US

(21) Appl. No.: 12/571,391

(22) Filed: Sep. 30, 2009

Related U.S. Application Data
(63) Continuation-in-part of application No. 11/554,364, filed on Oct. 30, 2006.
(60) Provisional application No. 60/767,546, filed on Jun. 21, 2006.

(51) Int. Cl. A61K 31/714; A61P 25/34; A61K 36/84; A61K 36/67; A61K 36/82

(52) U.S. Cl. 424/729; 514/52; 424/725

(57) ABSTRACT

A multi-component compound for the simultaneous treatment of nicotine addiction and the side effects of nicotine withdrawal, such as excessive appetite. The first component is a bivalent negative sulfur compound in an amount effective to control nicotine craving or the withdrawal symptoms resulting from nicotine withdrawal. The bivalent negative sulfur is selected from a group that includes, but is not limited to, alkyl sulfides, colloidal sulfur, hydropersulfides, organic thio compounds or their salts. The second component is a serotonin precursor, such as tryptophan or its derivative 5-HTP, which is used to assist the body in producing more serotonin which in turn suppresses appetite. The appetite suppressant(s) are combined with the bivalent negative sulfur compound(s) to provide a single compound that reduces nicotine craving and simultaneously suppresses increased appetite resulting from nicotine withdrawal. The compound alters the perceived taste of tobacco smoke such that it is no longer enjoyable.

### BACKGROUND OF THE INVENTION

1. Cross-Reference to Related Applications

This application is related to, and claims the benefit of, the now expired provisional patent application entitled “Smoking Cessation Treatment With Appetite Suppression”, filed Jun. 21, 2006, bearing U.S. Ser. No. 60/767,546 and naming Harlan Bieley, the named inventor herein, as sole inventor, the contents of which is specifically incorporated by reference herein in its entirety, and currently co-pending non-provisional patent application entitled “Smoking Cessation Treatment With Appetite Suppression”, filed Oct. 30, 2006, bearing U.S. Ser. No. 11/554,364 and naming Harlan Clayton Bieley, the named inventor herein, as sole inventor, the contents of which is specifically incorporated by reference herein in its entirety.

2. Technical Field

The present invention relates to treatments for individuals seeking to cease smoking. In particular, it relates to a method and treatment for cessation of smoking using a three-pronged approach. The compound includes ingredients which synergistically 1) reduces the craving for nicotine, 2) reduces appetite to eliminate the undesirable side effect of weight gain related to withdrawal from nicotine, and 3) alters the perceived taste of tobacco smoke to reduce enjoyment from smoking. The perceived taste change creates an unpleasant flavor that assists an individual attempting to cease smoking by reducing the pleasure derived from smoking. In addition, the compounds used by the invention also reduce hunger cravings and thereby help avoid unwanted weight gain.

3. Background

The well-known health risks and problems associated with tobacco smoking have caused numerous individuals to attempt to end their use of tobacco products. Unfortunately, this is at best a difficult task. It is common knowledge that tobacco products can be highly addictive due to the nicotine that is present in tobacco products. Every day, many individuals attempt to quit smoking by simply stopping their use of tobacco products. Unfortunately, due to the strong addictive nature of nicotine, the abrupt cessation of tobacco usage (i.e., quitting “cold turkey”) fails in most cases. Quite often, individuals require more than willpower alone to free themselves from nicotine addiction. It would be desirable to have a method of aiding an individual to cease smoking without having to rely on substantial levels of willpower.

In addition to the difficulty related to cessation of smoking, attempts to stop smoking often result in the creation of secondary problems related to smoking. In particular, a fairly common side effect that individuals experience when attempting to quit smoking is a substantial increase in appetite because they are craving food as a response to their anxiety when they attempt to stop smoking. The increase in appetite results in undesirable increases in body weight. In turn, the extra body weight can result in a variety of unwanted health problems, as well as an undesirable change in the individual's appearance. Further, the undesirable increase in body weight can have a detrimental effect on an individual's willpower when attempting to quit smoking, because the additional weight can be very frustrating to an individual. In fact, the anxiety created during the process of nicotine withdrawal may be so frustrating to people that they abandon their attempt to quit smoking. Weight gain often results as smokers increase the amount of food they eat as a way to relieve anxiety. It would be desirable to have a method of increasing an individual's chances of success by avoiding unwanted weight gain during the difficult process of overcoming addiction to nicotine.

Another substantial problem associated with smoking cessation is related to the enjoyment individuals obtain from smoking tobacco products. In particular, smokers enjoy the flavor of particular tobacco products. This loss of enjoyment is a substantial factor for individuals seeking to quit smoking. It would be desirable to have a method of assisting smokers in their efforts to quit smoking by eliminating the enjoyment associated with particular tobacco flavors.

To assist individuals attempting to quit smoking and free themselves of addiction to nicotine, a variety of methods have been tried. One such method has been the use of nicotine supplements to reduce nicotine craving by the individual. Nicotine supplements can take several forms. For example, a nicotine chewing gum has been developed which permits individuals to satisfy the body's craving for nicotine without damaging their lungs by inhaling tobacco smoke. Likewise, nicotine patches which adhere to an individual's skin for transdermal absorption of nicotine have also been developed. The object of nicotine supplements is to satisfy an individual's craving for nicotine, without the highly negative health consequences of inhaling tobacco smoke. While nicotine supplements protect the individual from the significant harm that smoking causes by satisfying the need to obtain nicotine without inhaling tobacco smoke, they do not overcome the individual's addiction to nicotine all of the time. In addition, once the chewing gum is discarded, or the transdermal patch is no longer used, the craving for tobacco products often returns. As a result, nicotine supplements solve only some of problems associated with smoking. It would be desirable to have a method of weaning an individual away from smoking tobacco, while at the same time minimizing the numerous undesirable side effects of a withdrawal from smoking.

More recently, alternative treatments which do not use nicotine supplements have been developed to assist individuals in breaking the tobacco habit. Nicotine is a neural chemical which attaches to specific cell receptor sites in the brain. Therefore, preventing its ability to attach to these cell receptor sites is one method used to interfere with the addictive qualities of nicotine. One such alternative treatment involves the use of chemical compounds which bind to these cell receptor sites, and thereby prevent nicotine from binding to those cell receptor sites. As a result, the nicotine based cravings of the individual are reduced. A number of anti-smoking compounds have been found to be effective for this purpose. These compounds include alkyl sulfides, colloidal sulfur, hydropersulfides, organic thio compounds or their salts. The preferred thio compounds are thioglycerols, thioglycols or their salts.

One example of a commercially available product containing at least one of these compounds is Sulfonil™. Sulfonil is described in U.S. Pat. No. 4,596,706 as a method of controlling craving for tobacco or controlling tobacco withdrawal symptoms. The product uses bivalent negative sulfur compounds which attach to the individual's cell receptor sites, and thereby prevent nicotine from binding to those same cell receptor sites.

By removing or reducing the ability of the nicotine to bind with cell receptor sites, the smoker can gradually eliminate the addiction to nicotine. However, while products such as Sulfonil help an individual to eliminate the nicotine craving, it does nothing to address the side effects of nicotine withdrawal. One such side effect is an excessive appetite that can result in an unwanted weight gain. As noted above, weight gain is not only deleterious to health, but it can also be an incentive to resume smoking. Therefore, in addition to addressing the chemical dependency of nicotine, a treatment for smoking cessation should also include a second function that reduces a smoker's tendency to gain weight. Weight gain, of course, discourages smokers from continuing the process of smoking cessation.

A third problem associated with smoking cessation is that smokers typically enjoy the taste of tobacco products and have particular preferences as to brands. In addition to the problems discussed above, a significant problem associated with smoking cessation relates to the enjoyment and gratification smokers receive from the flavor of a particular tobacco product. It would be of significant value to have a smoking cessation product that provided a third function, namely, altering an individual's perceived taste of a tobacco product. In particular, it would be desirable to have method of creating an undesirable perceived taste from tobacco smoke that would eliminate the enjoyment a smoker receives from tobacco products. If tobacco products produced an unpleasant taste, the smoker would then have a powerful incentive to stop smoking.

While the prior art has addressed some of the problems related to cessation of smoking, it has failed to provide a multimodal synergistic approach to smoking cessation that addresses the problem on these multiple fronts. In particular, the prior art has failed to provide a product that reduces nicotine cravings, reduces adverse side effects of nicotine withdrawal such as excessive weight gain, and alters the perceived taste tobacco smoke such that the change in perceived taste is not desirable and assists the withdrawal process by reducing the pleasure derived from smoking. It would be desirable to have a single product which is capable of addressing all three issues: 1) the basic problem of nicotine addiction, as well as subsequent withdrawal related side effects such as 2) excessive weight gain which in turn leads to other health problems, and 3) the desire to smoke because the taste makes it enjoyable. It would be desirable to have a single product that is capable of addressing all three issues. It has been found that by reducing or eliminating an individual's enjoyment of smoking by altering the perceived flavor of the tobacco product, the nicotine cravings and the weight gain side effects are easier to manage. As a result, it would be desirable to have a product that helps smokers escape from tobacco use by approaching the problem on three fronts: reducing chemical dependence on nicotine, avoiding weight gain, and eliminating the enjoyment smokers have while smoking tobacco.

### SUMMARY OF THE INVENTION

The present invention relates to a compound for the simultaneous treatment of nicotine addiction and undesirable side effects such as excessive appetite and weight gain that occur during the nicotine withdrawal process. In addition, the ingredients in the compound work synergistically to create an unexpected effect. Namely, the combination of ingredients in the compound alters the smoker's perception of taste such that tobacco smoke no longer tastes good. As a result, the compound produces three distinct effects. The first effect is to reduce cravings for nicotine, the second effect is to reduce cravings for food that results in weight gain, and the third effect is a change in the perceived taste of tobacco smoke on the part of the smoker that discourages the smoker relapsing.

The invention provides an improvement over the prior art due to the synergistic effect of the compound disclosed herein. The concept of synergy, coined by R. Buckminster Fuller, is well accepted with the word appearing in the English language and medical dictionaries. We use this word within the context that it is typically understood. For example, Steadman's Medical Dictionary defines synergy as: “The interaction of two or more agents or forces so that their combined effect is greater than the sum of their individual effects.” Fuller defined synergy to mean “behavior of integral, aggregate, or whole systems unpredicted by behavior of any of their components or subassemblies of their components taken separately from the whole.”

The first component of the compound uses at least one bivalent negative sulfur, in an amount effective to control the craving or the withdrawal symptoms resulting from nicotine withdrawal. The bivalent negative sulfur is selected from a group that includes, but is not limited to, hydropersulfides, alkyl sulfides, colloidal sulfur, organic thio compounds or their pharmaceutically acceptable salts. The most effective thio compounds have proven to be thioglycerols, thioglycols or their pharmaceutically acceptable salts combination of ingredients used herein work synergistically to not only alleviate cravings created by the tobacco withdrawal process, but adds another dimension to the smoking cessation process by altering the perceived taste of the tobacco products such that the perceived taste changes and becomes unpleasant. The combination of ingredients used by the invention works synergistically to create the unpleasant taste, which in turn eliminates a powerful incentive for a smoker to relapse.

The second component of the compound relates to cravings, appetite suppression, and control of blood sugar. In the preferred embodiment, appetite suppression is accomplished using amino acids derived from tryptophan, such as 5-HTP. 5-HTP and/or related tryptophan derivatives. These are known in the art to suppress appetite and cravings for certain carbohydrates when ingested. The appetite suppression compounds are combined and have synergy with the bivalent negative sulfur compound(s) to provide a single compound that produces the desirable effects of reducing nicotine craving while simultaneously suppressing increased appetite that is a result of nicotine withdrawal, and making tobacco smoke have a perceived unpleasant taste.

The third component is that the compound used by the invention produces a synergistic effect by altering the perceived taste of tobacco smoke such that the smoke has an unpleasant taste. The unpleasant taste bolsters the withdrawal process by reducing the enjoyment that may contribute to a smoker's decision to resume smoking.

These compounds are combined with suitable binders to form pharmaceutical capsules that can be administered orally. Alternatively, these compounds can be administered via injection, via transdermal cream, gel or patch, via gum or lozenge, transmucosally as a troche or suppository, as a liquid, powder to be mixed with a liquid, or by any other suitable means. In some administration methods, such as gum or lozenge, sweetening agents, such as sugar alcohols, can be added to sweeten the taste without effecting blood sugar levels. Sugar alcohols, such as xylitol, can be used in combination with gums, lozenges, powders, or liquids to provide sweetness with a lower carbohydrate level than a comparative amount of sugar. As a result, it will not elevate blood sugar levels as much as sugar will. In addition, stevia and agave may also be used as sweeteners. In addition, stevia leaf extract and agave nectar can both be useful in controlling blood sugar levels. Any other sweetening agents may also be included.

A therapeutic dosage range is approximately 120 to 480 milligrams per day of the bivalent negative sulfur, and the daily dosage can be broken up into smaller doses of that may be taken at different times of the day. Regarding 5-HTP, typical doses would run from a low dosage level of 50 milligrams to a high dosage level of 900 milligrams per day. It is important to note that the dosage level of each component of the compound can vary based on the size/weight of the individual in question, vary based on the tolerance level of that individual for components of the compound, or vary based on the intensity of the craving for nicotine or food. As a result, a small individual, or an individual sensitive to the compound, may achieve adequate results with small dosage levels of 5-HTP (e.g., less than 50 milligrams) per day. Likewise, a large individual, or an individual with a high tolerance level for the compound, may safely take more than what would typically be considered the maximum dosage of 5-HTP (e.g., greater than 900 milligrams) per day. It is important to note that the dosage level of each component of the compound can vary based on the size and/or weight of the individual taking the compound.

The treatment uses a compound that addresses nicotine craving, appetite control, and the perceived taste of tobacco smoke. The first component of the compound is designed to interfere with the ability of nicotine to attach to cell receptor sites, thereby reducing nicotine craving. The other component of the compound is directed to both nicotine craving and appetite control, thereby reducing excessive weight gain associated with withdrawal from nicotine addiction. The various components of the compound act together to assist an individual to quit smoking without the undesirable side effects of nicotine withdrawal, such as increased appetite and unwanted weight gain. Further, another component of the compound is that it provides an unexpected effect in that it alters the perceived taste of tobacco smoke.

An advantage of tryptophan and tryptophan derivatives derives from the fact that when the body processes a tryptophan derivative such as 5-HTP, the tryptophan derivative is used by the body as a serotonin precursor to increase serotonin levels. It is the increased serotonin level that decreases appetite and craving for food which lead to excessive weight gain. An added benefit provided by serotonin is that it tends to decrease the craving for nicotine. As a result of its general affect on craving, it not only helps suppress appetite which leads to unwanted weight gain, but it also works synergistically with the sulfur based compounds to decrease craving for nicotine.

### DESCRIPTION OF THE PREFERRED EMBODIMENT

Prior to discussing detailed aspects of the invention, a general overview will be provided. The invention provides an improvement over prior art sulfur-based nicotine withdrawal products which block nicotine from binding with cell receptor sites. The prior art has focused its attention on the use of a variety of sulfur based compounds to treat nicotine addiction. However, it has not placed significant emphasis on the side effects associated with nicotine withdrawal. In particular, a frequent side effect associated with smoking cessation is a marked increase in appetite. The increase in appetite often results in an individual having the health benefits associated with cessation of smoking offset by the health hazards produced by excessive weight gain. These are increased insulin levels that can lead to insulin resistance, increased blood pressure, and unfavorable changes in lipid profiles.

The present invention overcomes these problems by providing a single therapeutic compound which simultaneously reduces nicotine craving to assist an individual to cease smoking, alters the perceived taste of tobacco smoke to deter relapse, and simultaneously suppresses the individual's appetite to avoid unnecessary and undesirable side effects, such as weight gain, associated with the cessation of smoking. The compound provided by the present invention provides an unexpected effect. Namely, the ingredients of the compound work in conjunction with the suppression of nicotine cravings to further discourage smokers by altering the perceived taste of tobacco smoke. In particular, the compound alters the perceived taste of tobacco smoke such that it has an unpleasant taste that discourages relapse.

The invention takes advantage of known sulfur based compounds which interfere with the binding of nicotine with cell receptor sites. These compounds, and their method of use, are discussed in detail in U.S. Pat. Nos. 4,416,869 and 4,596,706, which are incorporated by reference herein in their entirety. Applicant's invention improves over these prior art inventions by adding compounds which are specifically designed to reduce the increased appetite which is caused by nicotine withdrawal. Further, the compounds selected to reduce appetite also enhance the body's ability to suppress nicotine cravings. As a result, an individual seeking to cease smoking will not have the additional problems generated by the common side effect of nicotine withdrawal, namely, increased appetite and weight gain. Applicant's invention provides a treatment regimen that works synergistically to overcome nicotine addiction while reducing the side effect of increased appetite that often occurs during nicotine withdrawal.

Having discussed the features and advantages of the invention in general, we turn now to a more detailed discussion of the figures.

As discussed above, the U.S. Pat. Nos. 4,416,869 and 4,596,706 teach a variety of sulfur based compounds which are useful in the treatment of nicotine addiction. In particular, these patents teach the use of bivalent negative sulfur compounds which are useful for reducing nicotine craving in individual's seeking to cease smoking tobacco products. However, these products do not effectively address side effects related to the cessation of smoking, most notably: unwanted weight gain due to increased appetite. An advantage of Applicant's invention is that it allows an individual to cease smoking without this undesirable side effect.

Applicant's invention provides a new compound which incorporates known compounds in the prior art (related to suppression of nicotine addiction) with a second compound that combined with the first compound to simultaneously suppress the unwanted increase in appetite which occurs during nicotine withdrawal. Applicant's invention uses derivatives of the amino acid tryptophan to reduce appetite so that an individual can cease smoking tobacco products without the added stress and health hazards related to excessive weight gain. The combination of these compounds produces an unexpected effect, namely, the perceived taste of tobacco smoke becomes unpleasant. As a result of this change in perceived taste, it becomes easier for a smoker to overcome a tobacco addiction.

In the preferred embodiment, both the compounds related to nicotine addiction, and the amino acids used for appetite suppression are combined for ingestion by the individual. Those skilled in the art will recognize that these compounds can be administered via a variety of methods, such as pills/capsule ingestion, liquid ingestion, intramuscular injection, intravenous injection, nasal spray, or via inhaler. When used as a liquid, the compound can be administered in combination with any suitable solution, liquid carrier, liquid medium, etc. The only requirement is that liquid selected is suitable for use with the components of the compound. However, while a variety of administration methods can be used (e.g., injection, transdermal patches, creams, gels, ointments, lozenges, gums, etc.), the preferred embodiment envisions the simple and convenient process of administering the compound via a pill or capsule.

In the preferred embodiment, the tryptophan derivative used by the Applicant is 5-hydroxytryptophan (“5-HTP”), which is a nutrient. It is derived from the amino acid L-Tryptophan. L-Tryptophan plays a vital role in our health. Tryptophan an essential amino acid for building proteins and enzymes, and serves as the precursor for serotonin, and the hydrogen carriers NADH and NADPH. 5-HTP functions as a precursor to serotonin (5-HT, 5-hydroxytryptamine). Serotonin, a neurotransmitter, plays an important role in regulation of mood, appetite, body temperature, and the secretion of various hormones. While serotonin does not readily cross the blood brain barrier, serotonin precursors such as L-tryptophan and 5-HTP can. Supplementation with these precursors increases levels of serotonin. In addition, 5-HTP is more efficient than L-tryptophan because it bypasses the rate-limiting step of serotonin synthesis (tryptophan hydroxylase).

Tryptophan has a variety of side effects when ingested by humans. It can be used as a mood-enhancer, and it can help individuals sleep. In fact, it has been widely used to treat insomnia and depression. It can increase pain tolerance, and it can also reduce appetite. An advantage of 5-HTP over some other appetite suppressants is that it has small molecule size. The 5-HTP accesses the brain from the bloodstream, and once in the brain, it can be converted into serotonin. It is the serotonin, created from the 5-HTP, acting on the different serotonin receptor sites, which ultimately acts as an appetite suppressant, and helps to reduce cravings for nicotine. A further advantage of 5-HTP over other potential appetite suppressants is that it is a naturally occurring compound which is produced in the body from tryptophan which is found in high-protein foods such as beef, chicken, fish, and/or dairy products. A normal variety of food products will include many items that are rich in tryptophan.

In the preferred embodiment, the dosage level of 5-HTP ranges from 50 mg per day up to 900 mg per day. 700 to 900 mg of 5-HTP would normally be considered a high dose. Of course, those skilled in the art will recognize that there are many factors that influence the appropriate dosage level for an individual. For example, the size, weight and tolerance of individuals can vary widely. Therefore, an appropriate dosage for one individual may not be safe for another. A large individual may have the ability to use dosage levels well in excess of 900 mg. Some individuals may also have high tolerance levels for particular compounds which will result in the ability to use high dosage levels. Of course the opposite will be true for individuals who are small or particularly sensitive to a given compound. In addition, other factors related to an individual may create higher or lower levels of appetite which would necessitate the change in dosage levels. As a result, unique factors related to each individual should be taken into account when determining the proper levels of each of the components of the compound.

In the preferred embodiment, the compound would be taken at least twice a day, to maintain stable levels of nicotine suppressant. In addition, it has been found that the appetite suppressant is more effective if taken before or after meals, because it is absorbed by the body more rapidly if taken on an empty stomach. As a result, an individual would preferably take the compound approximately an hour before meals, or approximately 2 hours after meals. Typically, an individual would take a dose at least twice a day. This would maintain relatively stable levels of nicotine suppressant throughout the day, and will also suppress hunger at the appropriate times.

Because 5-HTP is a precursor used by the body to produce serotonin which suppresses appetite, its use in combination with the aforementioned nicotine suppressant allows an individual to improve the chances of successfully overcoming a tobacco habit. This is because the individual will not have negative side effects such as increased weight gain, which may in fact frustrate the individual to the point where they resume smoking. Due to that, the individual is more likely to succeed when attempting to quit smoking. Of course, avoiding unnecessary weight gain provides many advantages for the overall health of the individual. In addition, because serotonin also inhibits cravings for nicotine, it will synergistically enhance the effect of the sulfur based compounds that are directed specifically at reducing nicotine cravings. Magnesium and 5-HTP work synergistically to decrease anxiety. Magnesium prolongs the effects of 5-HTP. An appropriate dose of magnesium is preferably 50-300 mg per day.

In addition to the use of 5-HTP to suppress appetite, other additives may be included in the compound to enhance weight control. For example, there are a number of nutritional supplements which enhance the body's ability to metabolize fat, such as vitamin B12, inositol, methionine, and choline. Additives such as this complement the appetite suppression by enhancing the body's ability to metabolize fat. Therefore, these additives would also help reduce the risk of weight gain during smoking cessation.

Inositol, in all of its forms, may be helpful in multiple ways. Higher doses may help increase GABA and reduce anxiety as does magnesium and 5-HTP. Inositol augments the effects of neurotransmitter release. While medical literature suggests that individuals may safely ingest 1-18 grams per day, the preferred embodiment envisions a dosage level of approximately 100 mg-2 gram/day.

Another effective additive is GABA at 100-2250 mg per day to decrease anxiety. GABA may be added directly, or GABA agonists, such as taurine 500-3000 mg per day), indirect GABA agonist such as L-theanine (100-800 mg per day). Magnolia officianalis (used in Seditol™) works on the GABA sites to reduce anxiety and on the adenosine A1 sites to lower anxiety and stress, magnesium malate (200-400 mg per day), vitamin B-6 (as pyridoxal hydrochloride and/or pyridoxal 5-phosphate, 25-50 mg per day), vitamin B6 is a cofactor in the synthesis of GABA from the enzyme glutamate acid decarboxylase (GAD), N-Acetyl Cysteine (NAC, 100-2000 mg per day) is a novel glutamate antagonist. It down-regulates glutamate activity by its involvement with the Cysteine-Glutamate Transporter. There is intracellular entry of Cysteine in exchange for transport of glutamate. It inhibits excitatory neurotransmission by activation of the XC-transporter, promoting metabotropic receptors an inhibiting glutamate. Also, Glycine, Niacinamide, Valerian root, kava kava, and green tea may be used. Green tea extract (Camilla sinensis) 60% catechins, 40% EGCG can be used at 300-600 mg per day. Taurine activates GABA A receptors in the hippocampus and stabilizes membranes. Taurine enhances brain GABA levels by inhibiting GABA transaminase and stimulating GAD. Anxiolytic effect has been observed in animals.

GABA is an inhibitory neurotransmitter that functions as: calming, stabilizing, antianxiety. GABA is the primary inhibitory neurotransmitter in the brain. GABA deficiency manifests as: anxiety, tremor, insomnia, tension, cardiac dysrhythmia, manic depression, OCD, adjustment disorders, phobias, and restlessness.

Another effective additive is Dopamine, which is a neurotransmitter. L-Tyrosine 100-500 mg a day as well as N-acetyl tyrosine 100-500 mg a day and L or DL-phenylalanine 100-500 mg a day will increase dopamine along with the B-Vitamins. The addition of the neurotransmitter precursor of dopamine may also help decrease cravings for certain carbohydrates like sweets, but also salt and fat. The L-phenylalanine form may work best.

Other optional additives can also be usefully combined with the compound. For example, alpha-lipoic acid may help reduce cravings for sweets by regulating blood sugar. Likewise, chromium picolinate, the chromium product, chromium polynicotinate™, biotin, and butyrate can be helpful in regulating blood sugar metabolism and may be included in the compound. Chromium and biotin synergistically improved glucose tolerance. Biotin 5-15 mg enhances the effects of enzymes involved in glucose metabolism. Chromium Polynicotinate may increase insulin receptor sensitivity and enhanced glucose uptake. Alpha lipoic acid stimulates the uptake of glucose via stimulation of GLUT-4 protein (primary glucose transporter in muscle, cardiac and fat cells). Alpha lipoic acid decreases appetite because it decreases AMP kinase in the brain and increases AMP kinase in peripheral tissues thus improving blood sugar regulation. Alpha lipoic acid turns on PGC Alpha that turns on GABA receptors. Butyrate improves insulin sensitivity. In addition to these nutritional supplements, vitamin B6 increases the efficacy of the conversion of 5-HTP to serotonin. Therefore, their inclusion in the compound may result in greater serotonin levels.

In the preferred embodiment, at least twice a day, the following doses should be taken:
1. 50-500 mg 5-HTP
2. 60-480 mg Thioglycerol
3. 25 mg-2 gram Choline in any of its forms (choline as choline citrate or phosphatidylcholine)
4. 25-100 mg Pantothenic Acid (as D-Calcium Pantothenate)
5. 200 mg-2 gram Inositol
6. 25-100 mg L-methionine
7. 50-500 mg Alpha Lipoic Acid
8. 25-50 mg Vitamin B-6 (as Pyridoxine HCl and/or Pyridoxal 5-Phosphate, Note: Vitamin B-6 (helps with conversion of 5-HTP to serotonin)
9. 25 mg-2 gram Vitamin C
10. 200 mcg-5 mg Folic Acid and/or Folinic Acid and/or L-Methylfolate, or any other forms of Folic acid
11. 50 mcg-1 mg Vitamin B12 (as Methylcobalamin or Cyanocobalamin)
12. 25-400 mg Magnesium Citrate and/or Magnesium Glycinate and/or Magnesium Malate

Optionally, the following may be added to the foregoing:
13. 100-500 mg L-Tyrosine
14. 100-500 mg N-acetyl tyrosine
15. 100-500 mg L or DL-phenylalanine
16. 50-800 mg L-theanine (for GABA and Dopamine modulation and to lower Norepinepherine)
17. 500 mg-10 gram Butyrate in the forms of sodium butyrate and/or sodium potassium butyrate and/or calcium magnesium butyrate
18. 365-720 mg in Seditol™ (A proprietary blend of patented natural plant extracts from magnolia officinalis and ziziphus spinosa)
19. 100-800 mg GABA or GABA agonists (such as taurine (50-3000 mg per day), indirect GABA agonists such as L-theanine (100-400 mg per day), Magnolia officianalis (used in Seditol™), magnesium malate, vitamin B-6 (as pyridoxal hydrochloride and/or pyridoxal 5-phosphate, 25-50 mg per day), N-Acetyl Cysteine (NAC, 50-2000 mg per day). Also, Glycine, Niacinamide, Valerian Root, Kava Kava, and Green Tea with Epigallocatechin Gallate (EGCG) binds to GABA receptors and reduces plasma cortisol levels in animals after social separation stress may be used)
20. 10-40 mg Vinpocetine

Choline, predominantly phosphatidylcholine (PC), maintains and reestablishes cellular membranes.

L-theanine does not directly increase GABA. It is a GABA modulator that dampens glutamate. It acts as a glutamate antagonist and also acts to modulate Dopamine. It also lowers Norepinepherine. As a glutamate antagonist that modulates GABA and Dopamine, L-theanine helps you stay calm.

Magnolia officinalis (found in Seditol™) works on the GABA A site to reduce anxiety, and also works on the Adenosine Al sites that can lower anxiety and stress.

Butyrate 500 mg-10 grams has been found to improve insulin sensitivity. It is preferably used in the form of sodium butyrate, sodium potassium butyrate, or calcium/magnesium butyrate. Other forms may also be used.

Those skilled in the art will recognize that while the aforesaid formula is preferred, changes can be made to the formula without losing its overall effectiveness. Two components of the compound are the most important. The first component is 5-HTP which is used to suppress appetite. In the preferred embodiment, this is administered in an approximate dosage of 200 mg. The second compound is Thioglycerol, which is a sulfur based compound that is used to block nicotine receptor cells. By blocking the receptor cells, nicotine cravings are reduced. The Thioglycerol is typically administered in an approximate dosage of 120 mg. The combination of these two components provides a compound that reduces nicotine cravings while simultaneously reducing appetite. In addition, the compound used by the invention has been found to have an unexpected effect. In particular, after taking the compound for approximately three days, users have been found to experience a change in the perceived taste of cigarette smoke, wherein the smoke takes on an unpleasant taste. The unpleasant taste bolsters the user during the withdrawal process facilitating smoking cassation, and they won't enjoy smoking if they relapse.

In addition to the two primary components, namely sulfur compounds used to block nicotine receptor cells, and the 5-HTP, which is used to increase serotonin levels, a variety of optional additional components can be used to enhance performance of the product. For example, weight suppression can be further enhanced through the use of Inositol, Vitamin B12 (as Methylcobalamin), L-methionine, and Choline. They can be used with one another to increase the body's ability to metabolize fat, and thereby helping the body to avoid weight gain. B vitamins, such as vitamin B-6 (as Pyridoxine HCl, or Pyridoxal 5-Phosphate, P-5-P), Pantothenic Acid (as D-Calcium Pantothenate), and Folic Acid are precursors to, or co-factors for neurotransmitters, which will decrease cravings for certain carbohydrates. Folic acid is also a natural serotonin agonist. It is a cofactor in serotonin synthesis. Alpha Lipoic Acid is helpful in stabilizing blood sugar levels. The Magnesium compounds may work synergistically with 5-HTP to reduce anxiety.

Those skilled in the art will recognize that as is the case with any pharmaceutical or neutraceutical, appropriate dosages will vary based on several factors. A person's weight, age, physical condition, etc., will all influence what the proper dosage for a particular individual should be. As a result, while the foregoing dosages are envisioned as an appropriate starting point, changes can be made to suit a particular individual's unique biochemical diversity.

Currently, there are more than 1 billion smokers on earth, and more than 400,000 Americans die annually of tobacco-related illnesses each year. More than 8 million Americans suffer from at least one serious illness caused by smoking; and almost 90 percent of all smokers begin at or before age 18 in the United States. The current invention seeks to reduce these numbers by providing a multimodal synergistic compound that reduces nicotine craving, that makes tobacco smoke taste unpleasant, and that helps with side effects of smoking cessation such as weight gain.

While the invention has been described with respect to a preferred embodiment thereof, it will be understood by those skilled in the art that various changes in detail may be made therein without departing from the spirit, scope, and teaching of the invention. For example, the compound used to suppress appetite can vary so long as it is suitable for its purpose, the amount of the appetite suppressant can vary based on an individual's physical requirements. The type of administration can vary from pill/capsule form to liquid form, injection, transdermal patch, creams, gels, ointments, gums, lozenges, etc. In addition, the dose and frequency of administration can vary based on the number of daily meals, severity of withdrawal systems, the weight and condition of the individual, etc. Accordingly, the invention herein disclosed is to be limited only as specified in the following claims. I claim:

What is claimed is:

1. A method of simultaneously treating tobacco/nicotine addiction, and weight control side effects caused by nicotine withdrawal by reducing the craving for nicotine, suppressing increases in appetite caused by nicotine withdrawal, and changing the perceived taste of tobacco smoke such that the smoker does not enjoy smoking, including the steps of:
using a compound having a nicotine craving suppressant with a second compound having at least one appetite suppressant into a treatment dosage, the compounds having approximately 25-2000 mg Choline, approximately 25-100 mg Pantothenic Acid (as D-Calcium Pantothenate), approximately 200-2000 mg Inositol, approximately 25-100 mg L-methionine, approximately 50-500 mg Alpha Lipoic Acid, approximately 25-50 mg Vitamin B-6 (as Pyridoxine HCl and/or Pyridoxal 5-Phosphate), approximately 25-2000 mg Vitamin C, approximately 200-5000 mcg Folic Acid and/or Folinic Acid and/or L-Methylfolate, approximately 50-1000 mcg Vitamin B12 (as Methylcobalamin or Cyanocobalamin), and approximately 25-400 mg Magnesium Citrate and/or Magnesium Glycinate and/or Magnesium Malate;
producing a synergistic effect with the compound that that alters the perceived taste of tobacco smoke such that it has an unpleasant taste; and
whereby the compound reduces the craving for nicotine, reduces cravings for food that result from the stress of withdrawal, and alters the perceived taste of tobacco smoke such that it is unpleasant.

2. A method, as in claim 1, including the additional step of:
reducing food cravings by increasing serotonin levels in the individual's body.

3. A method, as in claim 2, including the additional step of:
providing a serotonin precursor to assist individuals natural production of serotonin and individuals body.

4. A method, as in claim 3, including the additional step of:
using amino acids as the serotonin precursor.

5. A method, as in claim 4, including the additional step of:
using amino acids which include tryptophan derivatives.

6. A method, as in claim 5, including the additional step of:
using 5-HTP as at least one of the tryptophan derivatives.

7. A method, as in claim 6, including the additional step of:
administering the treatment dosage via pill/capsules, liquids, injections, transdermal patchs, creams, gels, ointments, transmucosal applications, gums, or lozenges.

8. A method, as in claim 2, including the additional steps:
using serotonin precursors derived from tryptophan to increase the serotonin levels; and
using at least one bivalent negative sulfur compound to reduce craving for nicotine.

9. A method, as in claim 8, including the additional step of:
using at least one bivalent negative sulfur compound from the group of hydropersulfides, alkyl sulfides, colloidal sulfur, organic thio compounds or their pharmaceutically acceptable salts.

10. A method, as in claim 9, wherein:
the bivalent negative sulfur compound or compounds are administered with a therapeutic dosage level of approximately 120 mg or more.

11. A method, as in claim 9, including the additional step of:
using thioglycerols, thioglycols or their pharmaceutically acceptable salts as the organic thio compounds.

12. A method, as in claim 11, including the additional steps of:
including approximately 50-500 mg of 5-HTP in the treatment dosage as a serotonin precursor; and
Including approximately 60-480 mg of thioglycerols, thioglycols or their pharmaceutically acceptable salts as a nicotine craving suppressant.

13. A method, as in claim 8, including the additional steps of:
using 5-HTP as the tryptophan derivative.

14. A method, as in claim 8, including the additional step of:
including one or more ingredients from the following group: L-Tyrosine, N-acetyl tyrosine, L or DL-phenylalanine, L-theanine, GABA, Butyrate in the forms of sodium butyrate and/or sodium potassium butyrate and/or calcium magnesium butyrate, taurine, L-theanine, Magnolia officianalis, magnesium glycinate, magnesium citrate, magnesium malate, vitamin B-6, N-Acetyl Cysteine, Glycine, Niacinamide, Valerian Root, Kava Kava, Green Tea with EGCG, and/or Vinpocetine.

15. A compound for simultaneous treatment of tobacco/nicotine addiction, and suppression of unwanted weight gain caused by nicotine withdrawal side effects, comprising:
a compound comprising approximately 25-2000 mg Choline, approximately 25-100 mg Pantothenic Acid (as D-Calcium Pantothenate), approximately 200-2000 mg Inositol, approximately 25-100 mg L-methionine, approximately 50-500 mg Alpha Lipoic Acid, approximately 25-50 mg Vitamin B-6 (as Pyridoxine HCl and/or Pyridoxal 5-Phosphate), approximately 25-2000 mg Vitamin C, approximately 200-5000 mcg Folic Acid and/or Folinic Acid and/or L-Methylfolate, approximately 50-1000 mcg Vitamin B12 (as Methylcobalamin or Cyanocobalamin), and approximately 25-400 mg Magnesium Citrate and/or Magnesium Glycinate and/or Magnesium Malate;
the compound having a nicotine craving suppressant, at least one appetite suppressant having at least one serotonin precursor to increase serotonin levels in an individual's body; and
the compound further acting synergistically to alter the perceived taste of tobacco smoke, such that the tobacco smoke has a perceived unpleasant taste;
wherein the nicotine cravings are reduced by the nicotine craving suppressant, the food cravings are reduced by increased serotonin levels in the individual's body, and the smoker is further assisted in withdrawal by the unpleasant taste of tobacco smoke.

16. A compound, as in claim 15, wherein:
the appetite suppressant is a tryptophan derivative.

17. A compound, as in claim 16, wherein:
the tryptophan derivative is 5-HTP.

18. A compound, as in claim 17, wherein:
the nicotine suppressant is a bivalent negative sulfur compound from the group of hydropersulfides, alkyl sulfides, colloidal sulfur, organic thio compounds or their pharmaceutically acceptable salts.

19. A compound, as in claim 18, wherein:
the appetite suppressant includes approximately 50-500 mg of 5-HTP; and
the nicotine suppressant includes approximately 60-480 mg of thioglycerols, thioglycols or their pharmaceutically acceptable salts.

20. A compound, as in claim 19, wherein:
the compound further comprises approximately 100-500 mg L-Tyrosine, approximately 100-500 mg N-acetyl tyrosine, and/or approximately 100-500 mg L or DL-phenylalanine.
